(19) United States
(12) Patent Application Publication
(10) Pub. No.: US 2022/0001048 A1
(43) Pub. Date: Jan. 6, 2022

(54) MATERIALS AND METHODS FOR PHOTOTHERMALLY SELF-DISINFECTING RESPIRATORS

(71) Applicants: Young-Shin Jun, St. Louis, MO (US); Srikanth Singamaneni, St. Louis, MO (US)

(72) Inventors: Young-Shin Jun, St. Louis, MO (US); Srikanth Singamaneni, St. Louis, MO (US)

(73) Assignee: Washington University, St. Louis, MO (US)

(21) Appl. No.: 17/366,440

(22) Filed: Jul. 2, 2021

Related U.S. Application Data
(60) Provisional application No. 63/047,757, filed on Jul. 2, 2020.

(51) Int. Cl. A61L 2/08; B01D 39/08; B01D 46/00; A41D 13/11; A62B 23/02

(52) U.S. Cl. CPC A61L 2/084; B01D 39/083; B01D 46/0028; B01D 46/0001; B01D 46/0063; A41D 13/1192; B01D 2239/10; B01D 2239/0618; B01D 2239/0627; B01D 2239/0622; B01D 2239/0428; B01D 2239/0442; B01D 2239/0478; A62B 23/025

(57) ABSTRACT

Self-sterilizing filtration materials are disclosed that include at least one polydopamine-functionalized layer. The at least one polydopamine-functionalized layer is configured to heat to a sterilization temperature when illuminated by light with a light intensity. The self-sterilizing filtration materials may be included in a filtering respirator mask. Methods of sterilizing filtration materials and filtering respirator masks containing at least one polydopamine-functionalized layer by exposing to sunlight are also disclosed. In addition, methods of producing a self-sterilizing filtering respirator mask by polydopamine-functionalizing at least one layer of the filtration material in an existing filtering respirator mask.

### CROSS-REFERENCE TO RELATED APPLICATIONS

This application claims the benefit of U.S. Provisional Application No. 63/047,757 filed Jul. 2, 2020, the contents of which are incorporated by reference in its entirety.

### STATEMENT REGARDING FEDERALLY SPONSORED RESEARCH & DEVELOPMENT

This invention was made with government support under grant CBET-1604542, awarded by the National Science Foundation. The government has certain rights in the invention.

### FIELD OF THE INVENTION

The field of the present disclosure generally relates to respirator filtration materials. Among the various aspects of the present disclosure is the provision of photothermally self-disinfecting filtration materials for use in respirators.

### BACKGROUND

Filtering facepiece respirators (FFRs) are a first line of defense in preventing infection through airborne particulate inhalation and in combating the ongoing pandemic of novel coronavirus disease (COVID-19). The sudden increase in global demand has created an unprecedented shortage of personal protection equipment (PPE) such as N95 masks (respirators). Because the virus is adsorbed on the respirator fabric during filtration, most N95 respirators are disposable and intended for single use. Enabling the safe reuse of N95 respirators would drastically mitigate their severe shortage problem, but the decontamination of used N95 respirators must be properly performed to minimize infection risk.

To reuse N95 respirators, hospitals are employing several disinfection treatments, including hydrogen peroxide vapor (HPV), ultraviolet light (UV) sterilization, and thermal sterilization. HPV has shown excellent microbicidal efficiency in treating N95 respirators, while minimally damaging their filtration and fit. Sterilizing an N95 respirator with HPV requires 125-480 minutes, including a conditioning phase, gassing phase, dwell phase, and aeration, and further requires specialized equipment to control the concentration and pressure of the vaporized H₂O₂ sterilant, so the HPV technique is usually limited to large hospitals. UV sterilization uses UVC light with a wavelength between 200 and 280 nm to kill or inactivate microorganisms by destroying their nucleic acid and disrupting their DNA. The disinfection efficacy of UV sterilization depends on the UV dose. With a proper dose, UV sterilization of N95 respirators has shown 99.9% virus inactivation, with minimal effects on filtration and fit. Although the decontamination system is less elaborate than that of HPV sterilization, UV sterilization still requires a UVC lamp and the UV dose must be carefully monitored. Furthermore, UVC light can harm the skin or eyes, so exposure precautions must be taken when using UV sterilization. Thermal sterilization is a traditional method that uses steam or hot air (dry heat) to denature the enzymes and structural proteins in microorganisms. Dry-heat sterilization is effective, but the >160° C. operating temperature and long exposure time associated with thermal sterilization significantly damage the filtration performance of N95 respirators. Respirators can be steamed in a microwave by heating them for 40-120 secs in an 1100-1250 W microwave oven. However, the steam's temperature and pressure should be carefully monitored during sterilization because the performance of N95 respirators could be compromised if steam reaches too high of a temperature under pressure.

### SUMMARY

In one aspect, a self-sterilizing filtration material is disclosed that includes at least one polydopamine-functionalized layer. The polydopamine-functionalized layer is configured to heat to a sterilization temperature of at least 70° C. when illuminated by light comprising at least one light wavelength ranging from about 200 nm to about 1000 nm and further comprising a light intensity of at least 1 kW m⁻² (1 sun). In some aspects, the at least one polydopamine-functionalized layer is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof. In some aspects, the at least one polydopamine-functionalized layer is further functionalized with FTCS such that the material has a higher surface hydrophobicity.

In another aspect, a filtering respirator mask comprising a self-sterilizing filtration material is disclosed that includes at least one polydopamine-functionalized layer. The polydopamine-functionalized layer is configured to heat to a sterilization temperature of at least 70° C. when illuminated by light comprising at least one light wavelength ranging from about 200 nm to about 1000 nm and further comprising a light intensity of at least 1 kW m⁻² (1 sun). In some aspects, the at least one polydopamine-functionalized layer is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof. In some aspects, the at least one polydopamine-functionalized layer is further functionalized with FTCS such that the material has a higher surface hydrophobicity.

In another aspect, a method of sterilizing a filtering respirator mask that includes at least one polydopamine-functionalized layer is disclosed. The method includes illuminating the at least one polydopamine-functionalized layer with a light at a light intensity sufficient to heat the filtering respirator mask to a sterilization temperature of at least 70° C. In some aspects, the at least one polydopamine-functionalized layer is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof. In some aspects, the at least one polydopamine-functionalized layer is further functionalized with FTCS such that the material has a higher surface hydrophobicity. In some aspects, the method may also include maintaining the illumination of the at least one polydopamine-functionalized layer for at least ten minutes. In some aspects, at least a portion of the light may include at least one light wavelength ranging from about 200 nm to about 1000 nm. In some aspects, the light intensity is at least 1 kW m⁻² (1 sun). In some aspects, the method may further include directing the light through at least one optical element to the at least one polydopamine-functionalized layer, the at least one optical element selected from the group consisting of a converging lens, a converging reflector, and any combination thereof.

In an additional aspect, a method of producing a self-sterilizing filtering respirator mask is disclosed. The method includes providing a filtering respirator mask that includes at least one layer of a filtration material and polydopamine-functionalizing at least one layer of the filtration material to produce at least one layer of a polydopamine-functionalized filtration material. The at least one layer of the polydopamine-functionalized filtration material is configured to heat to a sterilization temperature of at least 70° C. when illuminated by light comprising at least one light wavelength ranging from about 200 nm to about 1000 nm and further comprising a light intensity of at least 1 kW m⁻² (1 sun). In some aspects, the at least one layer of the filtration material is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof. In some aspects, polydopamine-functionalizing the at least one layer of the filtration material further comprises contacting the at least one layer of the filtration material with a dopamine solution comprising the dopamine in Tris-HCl with a pH of about 8.5, wherein contacting the at least one layer of the filtration material with the dopamine solution comprises spray coating the at least one layer of the filtration material with the dopamine solution or submerging the at least one layer of the filtration material in the dopamine solution. In some aspects, the method further includes additionally functionalizing the at least one layer of the polydopamine-functionalized filtration material to modify a surface hydrophobicity, wherein the additional functionalizing comprises subjecting the at least one layer of the polydopamine-functionalized filtration material to a fluoro-silanization process using FTCS. In some aspects, the method further includes applying at least one additional anti-viral coating to the at least one layer of the polydopamine-functionalized filtration material by applying a virucidal hydrophobic polycation to the at least one layer of polydopamine-functionalized filtration material. In some aspects, the at least one additional anti-viral coating may be a virucidal hydrophobic polycation comprising N,N-dodecyl,methyl-polyethylenimine.

Other objects and features will be in part apparent and in part pointed out hereinafter. Additional details of the disclosed materials and methods in various aspects are provided below.

### DESCRIPTION OF THE DRAWINGS

The patent or application file contains at least one drawing executed in color. Copies of this patent or patent application publication with color drawing(s) will be provided by the Office upon request and payment of the necessary fee.

The following drawings illustrate various aspects of the disclosure.

FIG. 1A is a schematic diagram illustrating a method of coating polydopamine photothermal moieties on fabric in situ in accordance with an aspect of the disclosure.

FIG. 1B is a diagram illustrating a chemical structure of a virucidal hydrophobic polycation (N,N-dodecyl,methyl-polyethylenimine) in accordance with an aspect of the disclosure.

FIG. 1C is a schematic diagram illustrating a process of virus inactivation on respirator fabrics coated with N,N-dodecyl,methyl-polyethylenimine virucide of FIG. 1C.

FIG. 2A is a series of temperature maps obtained from a series of IR images of a (tridecafluoro-1,1,2,2-tetrahydrooctyl)-trichlorosilane (FTCS)-PDA/BNC membrane exposed to 120 seconds of light irradiations at 1 kW m⁻² (1 sun).

FIG. 2B is a graph comparing the temperature profiles of pristine BNC and FTCS-PDA/BNC membranes exposed to 120 seconds of light irradiations at 1 kW m⁻² (1 sun) and 9 kW m² (9 sun).

FIG. 2C is a series of images of an FTCS-PDA/BNC membrane after exposure to E. coli for 1 hour.

FIG. 2D is a series of images of the FTCS-PDA/BNC membrane of FIG. 2C after exposure to solar light (1 kWm⁻²) for 10 minutes.

Those of skill in the art will understand that the drawings, described below, are for illustrative purposes only. The drawings are not intended to limit the scope of the present teachings in any way.

### DETAILED DESCRIPTION

In various aspects, photothermally self-disinfecting filtration materials for use in respirators and associated methods of photothermally disinfecting respirator filtration materials are disclosed herein. In some aspects, self-disinfecting N95 respirators may be produced by functionalizing commercially available N95 respirators with both photothermal and antiviral materials. The added photothermal materials will enable the N95 respirators to be periodically disinfected and to be further sanitized through simple sunlight exposure. Compared to hydrogen peroxide vapor (HPV), UV, and thermal sterilizations, photothermally-driven sterilization is an easy and scalable process for decontaminating N95 respirators under sustainable sunlight, and is readily implemented. Because the photothermally-driven sterilization methods described herein operate between about 70-80° C. to inactivate viruses and bacteria, photothermally-driven sterilization minimally deteriorates the filtration performance of N95 respirators over repeated sterilization cycles.

Commercially available N95 respirators are typically fabricated by stacking multiple layers of non-woven polypropylene and cotton/polyester fabrics. An FDA-approved four-layered N95 respirator typically consists of an outer layer of spun-bond polypropylene fabric for filtering large particulates, a second layer of cellulose/polyester fabric for filtering viruses, and a third layer of melt-blown polypropylene fabric for further isolation of viruses and an inner (fourth) layer of spun-bonded polypropylene fabric for breathability.

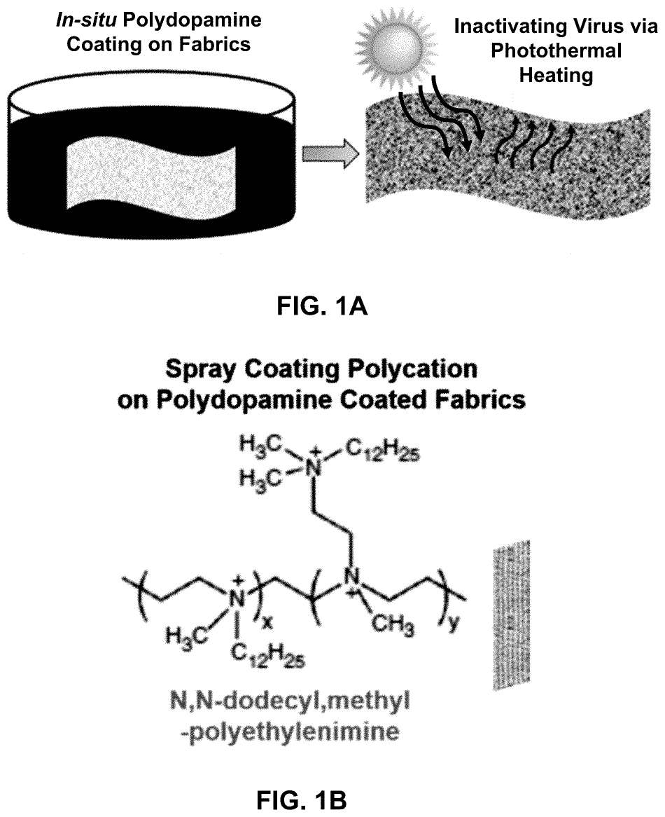

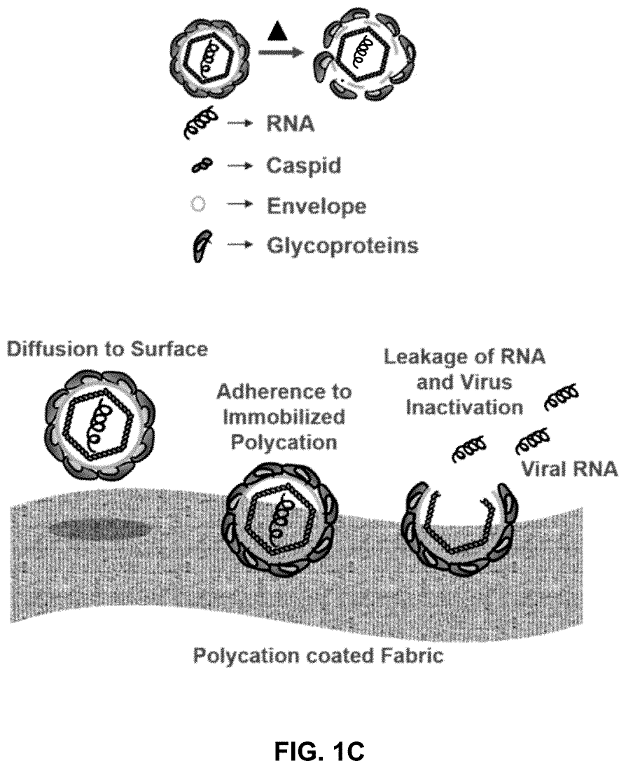

In various aspects, to enable reusing N95 respirators, the respirator may be functionalized with photothermal moieties using in situ polymerization of polydopamine (PDA) on the outer layer of the respirator fabric, including, but not limited to, a polypropylene outer layer. A schematic illustration of in situ polymerization of polydopamine (PDA) on a fabric is shown illustrated in FIG. 1A. PDA, formed by the oxidative self-polymerization of dopamine, is an important eumelanin-like biopolymer known for its versatile adhesion and surface modification capabilities. Structurally similar to eumelanin, PDA possesses many of the striking properties of naturally occurring eumelanin. For example, PDA can absorb 99% of incident photon energy over a broad solar spectrum and rapidly convert it into heat within tens of picoseconds, thus protecting living organisms against ultraviolet injury. Owing to PDA's superb photothermal ability and biodegradability, extensive efforts have been dedicated to utilizing PDA nanostructures as contrast agents for photothermal therapy.

The photothermal properties of PDA enhance the photothermal sensitivity of PDA-functionalized fabrics, membranes, and other materials, such that the PDA-functionalized materials achieve surface temperatures capable of killing microbes after exposure to sunlight. Without being limited to any particular theory, heat treatment induces structural changes in viral proteins and degrades viral RNA, thereby inactivating the virus. The applied temperature and the duration of heat treatment influence the achievement of the desired killing effects of heat treatment on viral protein as well as viral RNA.

In various aspects, filter membranes, fabric layers, or other elements of a respirator mask or other respirator device may be functionalized with PDA using an in situ polymerization method described below. The PDA-functionalized elements are capable of self-sterilizing by exposing the elements to sunlight or light at an intensity equivalent to sunshine for a duration sufficient to induce a surface temperature greater than a threshold sterilization temperature for at least a minimum sterilization time.

In various aspects, PDA-functionalized respirators can be disinfected under sunlight (either direct or concentrated by an inexpensive magnifying film) as illustrated in FIG. 1A, rendering the functionalized respirators reusable after disinfection. As illustrated in the examples below, the surface temperature of PDA-functionalized membranes increased to 77° C. within 20 seconds after exposure to simulated sunlight. As further illustrated in the examples below, a PDA-functionalized membrane coated with E. coli was sterilized after 20 minutes of exposure to sunlight. Without being limited to any particular theory, microbes such as E. coli bacteria and SARS-CoV-2 virus are killed at temperatures of about 70° C. The results described in the examples below indicate that the incorporation of PDA-functionalized materials in N95 respirators renders the respirators capable of self-disinfection under sunlight. In various aspects, the photothermal heating effects imparted by PDA functionalization inactivate viruses that are in direct contact with respirator surfaces as well as viruses that are not in direct contact with respirator surfaces.

In various aspects, the self-disinfection of PDA-functionalized materials may be influenced by any one or more factors including, but not limited to, the intensity of the sunlight, the duration of exposure to the sunlight, the ambient temperature at which the material is exposed to the sunlight, and any other relevant factor. Without being limited to any particular theory, the surface temperature of a PDA-functionalized material increases to a maximum temperature within a short time after initial exposure to sunlight, and this maximum temperature is maintained without further increase during sustained exposure to sunlight. In addition, the maximum surface temperature achieved by a material is proportional to the intensity of the sunlight to which the material is exposed. By way of non-limiting example, if the intensity of sunlight is less than a typical value of sunlight intensity 1 kWm⁻², a longer sunlight exposure time may be needed to ensure self-disinfection of the PDA-functionalized material.

In various aspects, the PDA-functionalized material may be any material used in the construction of masks without limitation. Non-limiting examples of suitable PDA-functionalized materials include non-woven polypropylene fabrics, cotton fabrics, polyester fabrics, spun-bond polypropylene fabrics, cellulose fabrics for filtering viruses, melt-blown polypropylene fabrics, and any other suitable mask material. In some aspects, the outermost layer of the mask is constructed of a PDA-functionalized material and the heating of this outermost layer during self-disinfection is sufficient to sterilize the underlying layers that are not PDA-functionalized. In other aspects, at least a portion of the underlying inner layers of the mask is also PDA-functionalized. In various additional aspects, PDA-functionalized materials may further include other respirator materials including, but not limited to, replaceable respirator filter materials and replaceable ventilator filter materials that may be removed, sterilized under light, and returned to the respirator or ventilator for re-use.

In various aspects, the various layers of the mask may be PDA-functionalized in situ as they are positioned within an assembled mask. In various other aspects, individual layers of the mask may be PDA-functionalized prior to assembly of the layers into a mask.

In various aspects, a material layer is PDA-functionalized using any known method without limitation. In one aspect, the material layer is PDA-functionalized using an in situ self-polymerization method. One non-limiting example of an in situ self-polymerization method is described in Acc. Chem. Res. 2014, 47, 3541-3550, the content of which is incorporated by reference in its entirety. The in situ method includes contacting the material layer to be PDA-functionalized with a solution of dopamine in Tris-HCl with a pH of about 8.5 for a period of time sufficient to allow the dopamine monomers to self-polymerize on the material layer. In some aspects, the material layer is submerged in the dopamine solution to PDA-functionalize the material layer. In other aspects, dopamine solution is spray-coated onto the material layer at least once to PDA-functionalize the material layer. In various aspects, at least one or more process parameters may be modulated to achieve the desired PDA-functionalization of the material layers. Non-limiting examples of suitable process parameters of the in situ self-polymerization process include concentration of dopamine solution, immersion time of the material in the dopamine solution, amount of dopamine solution spray-coated onto the material layer, pH of dopamine solution, and any other relevant process parameter.

In some aspects, one or more material layers of the mask may be further functionalized to adjust surface hydrophobicity of the material layer. Any known method of hydrophobic surface functionalization may be used without limitation including, but not limited to, a facile fluoro-silanization method using (tridecafluoro-1,1,2,2-tetrahydrooctyl)-trichlorosilane (FTCS). A non-limiting example of a suitable fluoro-silanization method is described in Langmuir 2011, 27(5), 1930-1934, the content of which is incorporated by reference in its entirety.

In various aspects, the PDA-functionalized material, when exposed to light including wavelengths within at least a portion of the PDA absorption spectrum, is heated. Without being limited to any particular theory, if exposed to light within the PDA absorption spectrum at sufficient intensity, the temperature of the PDA-functionalized material layer will increase to a temperature sufficient to sterilize the PDA-functionalized material layer and/or adjacent or underlying material layers of the mask. The PDA absorption spectrum includes light at wavelengths ranging from about 200 nm to about 1000 nm.

In some aspects, natural sunlight or sunlight that is intensified using optical elements including, but not limited to converging lenses or converging reflectors, may be directed onto the PDA-functionalized material layers to accomplish self-sterilization. In other aspects, artificial light that includes wavelengths within at least a portion of the PDA absorption spectrum may be used to accomplish self-sterilization.

In various aspects, the radiation intensity of the light used to self-sterilize the mask containing PDA-functionalized material layers is sufficiently high to heat the mask to sterilization temperatures of at least 70° C. In other aspects, the sterilization temperature is at least 72° C., at least 74° C., at least 76° C., at least 78° C., at least 80° C., at least 82° C., at least 84° C., at least 86° C., at least 88° C., at least 90° C., at least 92° C., at least 94° C., at least 96° C., at least 98° C., and at least 100° C.

Without being limited to any particular theory, the radiation intensity of the light used to self-sterilize the mask is influenced by the proportion of light falling within the PDA absorption spectrum. In various aspects, sunlight at an intensity of at least 1 kW m⁻² (1 sun) is used to sterilize the mask containing PDA-functionalized material layers. In other aspects, the mask is sterilized using sunlight at an intensity of at least 2 kW m⁻² (2 suns), at least 3 kW m⁻² (3 sun), at least 4 kW m⁻² (4 sun), at least 5 kW m⁻² (5 suns), at least 6 kW m⁻² (6 suns), at least 7 kW m⁻² (7 suns), at least 8 kW m⁻² (8 sun), at least 9 kW m⁻² (9 sun), and at least 10 kW m⁻² (10 suns).

As demonstrated in the examples below, upon exposure to sunlight, the surface temperature of a PDA-functionalized material layer rapidly increases to a maximum surface temperature that is maintained as long as the material is exposed to the sunlight. The maximum temperature achieved is proportional to the sunlight intensity illuminating the surface of the PDA-functionalized material.

In various aspects, the mask is exposed to the light during sterilization in order to maintain the temperature of the material layers of the mask at a temperature sufficiently high to kill any microbes present within the material layers. Without being limited to any particular theory, the amount of time at which the mask is exposed to light varies depending on the maximum temperature achieved by the PDA-functionalized materials illuminated by the light. In various aspects, the mask may be exposed to light for an exposure time ranging from about 5 minutes to about 2 hours. In other aspects, the mask may be exposed to light for an exposure time of at least 10 min, at least 20 min, at least 30 min, at least 40 min, at least 50 min, at least 60 min, at least 70 min, at least 80 min, at least 90 min, at least 100 min, at least 110 min, and at least 120 min.

In various other aspects, at least one additional antiviral coating may be applied over the PDA-functionalized respirator materials. The antiviral coatings may include any coating material capable of inactivating viruses by direct contact. Any known antiviral composition may be used as an antiviral coating including, but not limited to, virucidal hydrophobic polycation. Non-limiting examples of suitable virucidal hydrophobic polycations include N,N-dodecyl,methyl-polyethylenimine, the structure of which is shown illustrated in FIG. 1B. The antiviral coating may be applied over the PDA-functionalized respirator materials using any known suitable method without limitation including, but not limited to, spray coating.

As illustrated in FIG. 1C, viral particles adhere to the N,N-dodecyl,methyl-polyethylenimine coating via hydrophobic and electrostatic interactions. As a result, the viral particles disintegrate, accompanied by RNA leakage and loss of infectivity. The rigid and erect hydrophobic polycationic chains of the antiviral coating act as needles and rupture the viral lipid envelopes, killing the virus. In various aspects, the synergistic effects of the antiviral coating and the photothermal inactivation of the virus by the PDA-functionalized respirator materials concurrently disinfect the respirators and provide for multiple reuses.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Surface Temperature Profiles of PDA-Functionalized Membranes

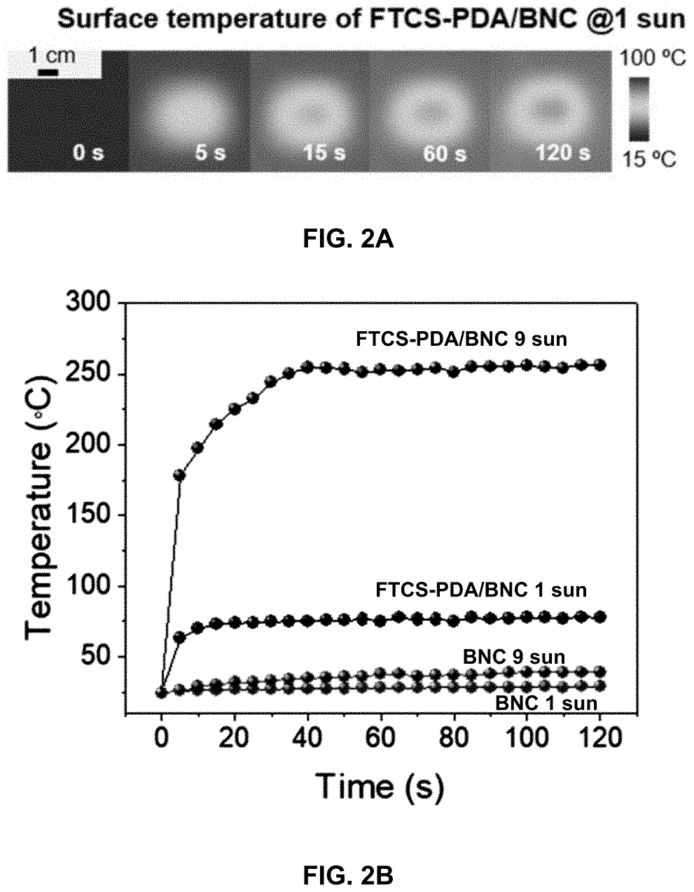

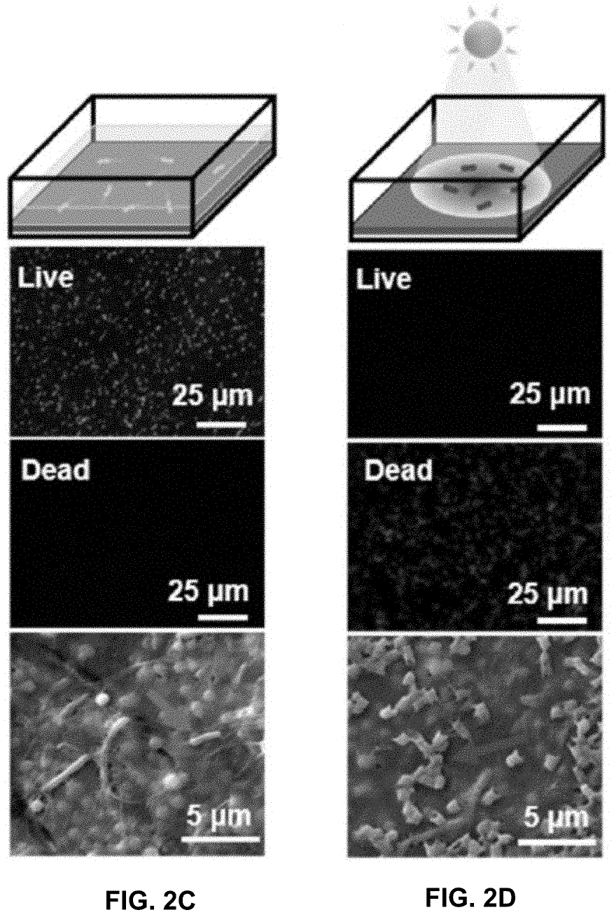

To evaluate the photothermal performance of PDA-incorporated polymeric fabrics, the following experiments were conducted. A polymeric bacterial nanocellulose (BNC) membrane was subjected to the in situ PDA polymerization method described above to produce a PDA-functionalized fabric (tridecafluoro-1,1,2,2-tetrahydrooctyl)-trichlorosilane (FTCS)-PDA/BNC). The PDA-functionalized membrane, as well as a control membrane (BNC) were subjected to illumination by a sunlight simulator at intensities of 1 kW m² (1 sun) and 9 kW m² (9 suns). The surface temperature evolution of the membranes was monitored using IR imaging. FIG. 2A is a series of surface temperature maps derived from the IR images showing that the PDA-functionalized membrane exposed to the equivalent intensity of sunlight (1 sun) reached a surface temperature of 77° C. within 20 seconds after exposure to the simulated sunlight. FIG. 2B is a graph comparing the surface temperatures of the PDA-functionalized membrane and control membrane during exposure to the simulated sunlight at intensities of 1 sun and 9 suns, illustrating that the PDA-functionalized membrane temperature increases to about 250° C. within 30 seconds after exposure to the simulated sunlight at an intensity of 9 suns.

### Example 2: Photothermal Disinfection of PAD-Functionalized Membranes

To test the disinfection performance of the PDA-functionalized fabrics, the following experiments were conducted. The PDA-functionalized membrane of Example 1 was exposed to E. coli, a microbe that can be killed at temperatures of about 70° C. Without being limited to any particular theory, the photothermal disinfection of E. coli as described below is thought to be representative of photothermal disinfection of other microbes including SARS-CoV-2 virus, that is inactivated within 5 min exposure to surface temperatures of 70° C. FIG. 2C is a series of images demonstrating that the PDA-functionalized membrane harbored live E. coli after exposure.

The PDA-functionalized membrane with live E. coli was then exposed to light at an intensity of 1 sun for 20 minutes. FIG. 2D is a series of images of the PDA-incorporated membrane after exposure to the light, demonstrating that the E. coli were successfully killed by the light exposure.

What is claimed is:

1. A self-sterilizing filtration material comprising at least one polydopamine-functionalized layer, wherein the polydopamine-functionalized layer is configured to heat to a sterilization temperature of at least 70° C. when illuminated by light comprising at least one light wavelength ranging from about 200 nm to about 1000 nm and further comprising a light intensity of at least 1 kW m⁻² (1 sun).

2. The material of claim 1, wherein the at least one polydopamine-functionalized layer is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof.

3. The material of claim 1, wherein the at least one polydopamine-functionalized layer is further functionalized with FTCS to enhance surface hydrophobicity.

4. A filtering respirator mask comprising a self-sterilizing filtration material, the self-sterilizing filtration material comprising at least one polydopamine-functionalized layer, wherein the polydopamine-functionalized layer is configured to heat to a sterilization temperature of at least 70° C. when illuminated by light comprising at least one light wavelength ranging from about 200 nm to about 1000 nm and further comprising a light intensity of at least 1 kW m⁻² (1 sun).

5. The mask of claim 4, wherein the at least one polydopamine-functionalized layer is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof.

6. The mask of claim 4, wherein the at least one polydopamine-functionalized layer is further functionalized with FTCS to enhance surface hydrophobicity.

7. The mask of claim 4, wherein the at least one polydopamine-functionalized layer is further functionalized with at least one additional anti-viral coating.

8. A method of sterilizing a filtering respirator mask comprising at least one polydopamine-functionalized layer, the method comprising illuminating the at least one polydopamine-functionalized layer with a light at a light intensity sufficient to heat the filtering respirator mask to a sterilization temperature of at least 70° C.

9. The method of claim 8, wherein the at least one polydopamine-functionalized layer is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof.

10. The method of claim 8, wherein the at least one polydopamine-functionalized layer is further functionalized with FTCS to enhance surface hydrophobicity.

11. The method of claim 8, further comprising maintaining the illumination of the at least one polydopamine-functionalized layer for at least ten minutes.

12. The method of claim 8, wherein at least a portion of the light comprises at least one light wavelength ranging from about 200 nm to about 1000 nm.

13. The method of claim 8, wherein the light intensity is at least 1 kW m² (1 sun).

14. The method of claim 8, wherein illuminating the at least one polydopamine-functionalized layer with the light at the light intensity further comprises directing the light through at least one optical element to the at least one polydopamine-functionalized layer, the at least one optical element selected from the group consisting of a converging lens, a converging reflector, and any combination thereof.

15. A method of producing a self-sterilizing filtering respirator mask, the method comprising:
a. providing a filtering respirator mask comprising at least one layer of a filtration material; and
b. polydopamine-functionalizing the at least one layer of the filtration material to produce at least one layer of a polydopamine-functionalized filtration material, wherein the at least one layer of the polydopamine-functionalized filtration material is configured to heat to a sterilization temperature of at least 70° C. when illuminated by light comprising at least one light wavelength ranging from about 200 nm to about 1000 nm and further comprising a light intensity of at least 1 kW m⁻² (1 sun).

16. The method of claim 15, wherein the at least one layer of the filtration material is selected from the group consisting of a non-woven polypropylene fabric layer, a cotton fabric layer, a polyester fabric layer, a spun-bond polypropylene fabric layer, a cellulose fabric layer, a melt-blown polypropylene fabric layer, and any combination thereof.

17. The method of claim 15, wherein polydopamine-functionalizing the at least one layer of the filtration material further comprises contacting the at least one layer of the filtration material with a dopamine solution comprising the dopamine in Tris-HCl with a pH of about 8.5, wherein contacting the at least one layer of the filtration material with the dopamine solution comprises spray coating the at least one layer of the filtration material with the dopamine solution or submerging the at least one layer of the filtration material in the dopamine solution.

18. The method of claim 15, further comprising additionally functionalizing the at least one layer of the polydopamine-functionalized filtration material to modify a surface hydrophobicity, wherein the additional functionalizing comprises subjecting the at least one layer of the polydopamine-functionalized filtration material to a fluoro-silanization process using FTCS.

19. The method of claim 15, further comprising applying at least one additional anti-viral coating to the at least one layer of polydopamine-functionalized filtration material by applying a virucidal hydrophobic polycation to the at least one layer of polydopamine-functionalized filtration material.

20. The method of claim 19, wherein the virucidal hydrophobic polycation comprises N,N-dodecyl,methyl-polyethylenimine.
